(19) United States
(12) Patent Application Publication
(10) Pub. No.: US 2026/0123852 A1
(43) Pub. Date: May 7, 2026

(54) ELASTOMER SUPPORT ASSEMBLY FOR USE IN CONNECTION WITH A WEARABLE DEVICE

(71) Applicant: Eoin Everard, Kilkenny (IE)

(72) Inventor: Eoin Everard, Kilkenny (IE)

(21) Appl. No.: 18/938,518

(22) Filed: Nov. 6, 2024

(51) Int. Cl. A61B 5/11; A61B 5/00

(52) U.S. Cl. CPC A61B 5/1114; A61B 5/0002; A61B 5/4561; A61B 5/6831; A61B 5/742; A61B 5/7455; A61B 5/746; A61B 5/7475; A61B 2562/0261

(57) ABSTRACT

Disclosed is an elastomer support assembly for use in connection with a wearable device. The elastomer support assembly includes an elastomer support and a sensor detachably integrated within the elastomer support. In one embodiment, the support is attached to the wearable device and adhered to a lumbar spine of a user when the wearable device is worn by the user for allowing sensors to detect lower back movement of the user.

### FIELD OF THE INVENTION

The present invention relates to posture monitoring device and more particularly relates to elastomer support assembly for use in connection with a wearable device for detecting lower back movement of a user.

### BACKGROUND OF THE INVENTION

Existing systems such as sensors embedded in garments or belts may not conform to the natural curvature of the lower back, specifically the lordotic region, leading to inaccurate detection of user's low back position or lumbar spine position. Such misalignment may result in inconsistent feedback, thereby reducing the reliability of the data gathered during motion in the lower back either when sitting, lifting or exercising.

Further, existing systems relying on external belts or garments for sensor attachment often lacks adaptability to various body shapes and sizes. Another drawback is the inconvenience associated with static design of such wearable. This requires users to wear specific garments or belts, which can be cumbersome and impractical for regular use. Likewise, the likelihood of consistent use may be reduced.

Existing systems often compensate for poor sensor attachment by increasing the sensitivity of the sensors to ensure some level of accuracy despite an inability to replicate the subtle movements of the lumbar spine. However, this increased sensitivity can result in false positives, where normal movements or slight shifts are interpreted as significant spinal misalignments. This issue exacerbates the unreliability of these systems, while providing users with incorrect feedback and potentially leading to unnecessary interventions or adjustments in posture when none are required. On the other hand, if the sensor in the garment does not stay connected to the lower back (Lumbar Spine), then subtle but important movements of the lower back may be missed. Therefore, it is necessary that the sensors stay in congruency with the lower back at all times and replicates the exact movements of the low back, however subtle.

Belts and garments are often designed with one-size-fits-all solutions that do not account for individual anatomical differences, particularly in the lower back's lordotic curvature. Without a system that naturally adjusts to these variations, users with different body types may experience discomfort and ineffective sensor performance, as the sensor may not remain tight against the skin, further compromising data accuracy.

Furthermore, a critical issue in existing systems is the risk of sensor detachment during motion. Belts and garments may become loose or shift position, especially during activities involving bending, twisting, or dynamic movement.

In conclusion, without the described improvements, existing sensor-based systems for monitoring spinal movements continue to face significant limitations. These include inaccurate readings, false positives, and inconvenient application, all of which reduce the reliability and practicality of the system in real-world scenarios.

Hence, there is a need for a wearable sensor device that overcomes the aforementioned drawbacks.

### BRIEF SUMMARY OF THE INVENTION

In an exemplary embodiment, the present invention discloses a flexible component for attaching to a body-wearable device that securely adheres to the back of a user while ensuring optimal contact and stability.

In another exemplary embodiment, the present invention discloses a flexible component including but not limited to foam, silicone or rubber, for securely holding a sensing component.

In an exemplary aspect, the present invention discloses a flexible component for securely holding a sensing component to detect spinal movement of a user.

In another exemplary aspect, the present invention integrates a sensor within a flexible component in such a way that the sensor is able to detect even the most subtle movements of the spine or lower back, while allowing for precise and accurate monitoring thereof.

In an exemplary embodiment, the present invention discloses integrating a flexible component comprising a sensor into a body-wearable device for detecting the change in posture of the user.

In another exemplary embodiment, the present invention discloses a body-wearable position monitoring device that mimics exact movements of the user's lumbar spine for accurate posture detection.

In certain embodiment, the present invention enables secure and stable attachment of the sensor device to a user's lower back using a body-wearable means.

In further embodiment, the present invention allows a flexible component of a body-wearable device to maintain consistent contact with the skin while reducing the risk of sensor detachment or misalignment during movement.

In an exemplary aspect, the present invention enhances comfort and sensor accuracy by utilizing an open-cell foam structure that adapts to various body shapes or lordotic curves during any movement and in any position.

In another exemplary aspect, the present invention incorporates an initial calibration process through a mobile or computer application to establish a baseline for accurate movement detection.

In yet another exemplary aspect, the present invention allows easy attachment, removal, and replacement of sensors from the support member without additional belts, straps, or garments.

In further aspect, the present invention creates a dynamic system capable of real-time posture monitoring and adaptation based on user preferences or automatic recalibration.

In certain aspect, the present invention comprises a mobile application to recalibrate the sensor according to the user's desired spinal position.

In another aspect, the present invention discloses a body-wearable device, which includes but not limited to belts, garments, vests, braces, and other wearable apparatuses, that enhances usability and comfort across various activities.

In an exemplary embodiment, the present invention provides haptic feedback to user upon detecting poor posture for immediate correction by the user.

In certain aspect, the present invention facilitates wireless communication between the sensor and a mobile device for real-time visualization of the user's posture.

In further aspect, the present invention discloses a mobile application for real-time posture visualization and feedback using digital representations of the user's spinal position.

According to an embodiment of the present invention, an elastomer support assembly for use in connection with a wearable device is disclosed. The elastomer support assembly includes an elastomer support and a sensor detachably integrated within the elastomer support. In one embodiment, the support is attached to the wearable device and adhered to a lumbar spine of a user when the wearable device is worn by the user for allowing sensors to detect lower back movement of the user.

### BRIEF DESCRIPTION OF DRAWINGS

The present invention will become clearly understood to those of ordinary skill in the art when descriptions of exemplary embodiments thereof are read with reference to the accompanying drawings.

FIG. 1 is a disassembled view of a belt and posture monitoring device according to an embodiment of the present invention.

FIG. 2 is a front view of the belt of FIG. 1 showing the placement method.

FIG. 3 is a rear view of the belt of FIG. 1.

FIG. 4 is a perspective view of a belt with posture monitoring device of FIG. 1.

FIG. 5 is a schematic view of the posture monitoring device of FIG. 1 to be positioned within the belt.

FIG. 6 is a side view of the belt of FIG. 2.

FIG. 7 shows different views of a silicone housing of a posture monitoring device.

FIG. 8 depicts a user standing straight, wearing a belt integrated with elastomer support assembly, and a screen of the user's smart device is shown.

### DETAILED DESCRIPTION OF THE INVENTION

The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention. For a more complete understanding of the present invention, reference is now made to the following descriptions taken in conjunction with the accompanying drawings.

In an exemplary embodiment, the present invention disclosed herein is a flexible component, hereinafter referred to as “support member” “sensor support member” or “flexible molded component”, designed to incorporate a sensor and to attach to a user's lower back for detecting lower back movement of the user. The lower movement may also be referred to as spine movement, low back movement, lumbar spine movement or various lordotic positions. The term movement may also be interchangeably referred to as, change in position or posture.

In another exemplary embodiment, the present invention disclosed herein is a body-wearable posture monitoring device designed to incorporate the aforementioned flexible component, which is then attached to a user's lower back for detecting the low back movement of the user. The sensor support member, i.e. the flexible component, is made of foam which is manufactured using either polyurethane (PU) foam or silicone foam, a rubber or any other foam or material that works in a similar manner. Using open-cell foam allows the support member to adapt to various body shapes, ensuring a comfortable fit and good contact with the body.

In an exemplary aspect, the flexibility of the support member allows the sensor to detect even the most subtle movements of the spine or lower back, while allowing for precise and accurate monitoring of the movements. In specific, the sensor support member mimics the actions or movements of the low back of a user due to the presence of foam material. The adaptability of sensor support member more closely to the lumbar spine positioning helps achieve accurate detection of the user posture by the sensor.

This flexibility makes it ideal for applications requiring close body contact and responsiveness to movement, enhancing the overall comfort and effectiveness of the sensor.

In various embodiment's, the support member is designed to conform to the natural lordotic curvature of the lower spine for ensuring secure and stable attachment with the body-wearable means. The flexibility of these materials used for constructing support member allows the sensors to maintain consistent contact with the skin, reducing the risk of detachment or misalignment during movement.

The open-cell foam structure allows the support member to adapt under pressure to different body shapes, providing comfort and ensuring a snug fit, which is essential for accurate sensor readings. The silicone variation, known for its flexibility and adherence properties, particularly excels in securing the sensor to the lower back, where movements are often more pronounced and harder to track with traditional belt or garment systems.

An important aspect of the device is the initial calibration process. Once the device is worn by the user, the user calibrates the device through a mobile or computer application. This calibration ensures that the sensor detects movements relative to a known baseline or zero point, eliminating inconsistencies in feedback and allowing the sensor to work accurately during the user's activities.

Additionally, the silicone or foam attachment is detachable, allowing users to easily attach, remove, or replace the sensors as needed without the need for additional belts, straps, or garments. This detachable feature also enhances user comfort and convenience.

In an exemplary embodiment, the device is said to be dynamic, since the device is capable of monitoring the user's posture in real time, and adapt the ideal spinal position based on the user's desired posture or, alternatively, automatically based on predetermined factors. The device is capable of functioning continuously, regardless of the user's movements or activities. This functionality is preferably achieved through the integration of the posture sensor and the ECU, which work in tandem to consistently capture the spinal position and provide immediate feedback as needed.

Since the device is further enhanced by the mobile application which is accessible via the mobile device, the device allows for recalibration of the posture sensor in accordance with the user's desired spinal position, or automatically based on predetermined factors. This recalibration feature enables the user to reset the zero value of the sensors at any time, while allowing the setting of a new ideal posture or spinal position based on specific activities, such as exercises.

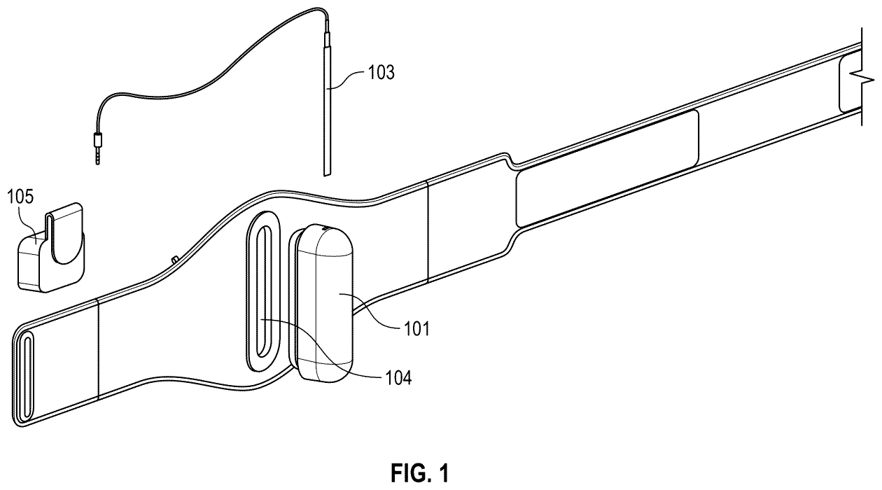

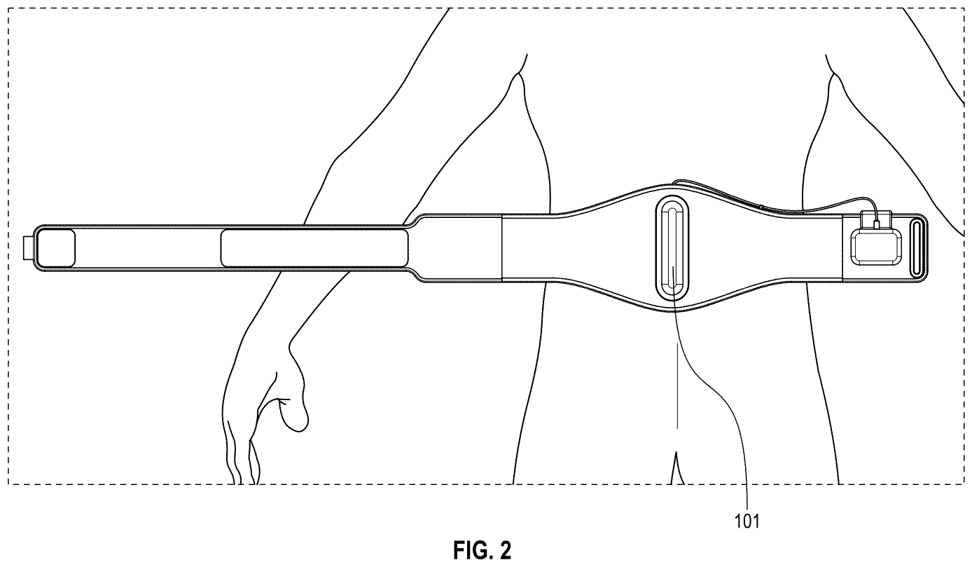

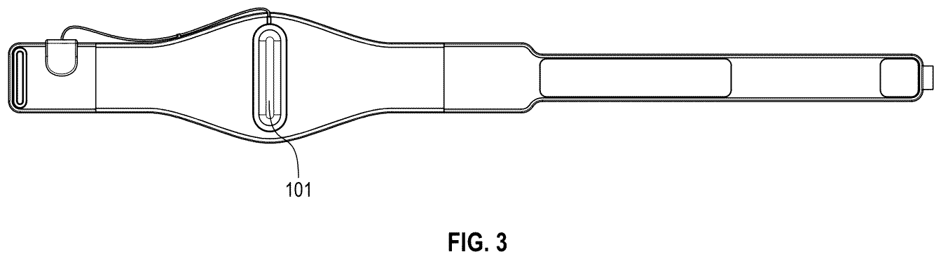

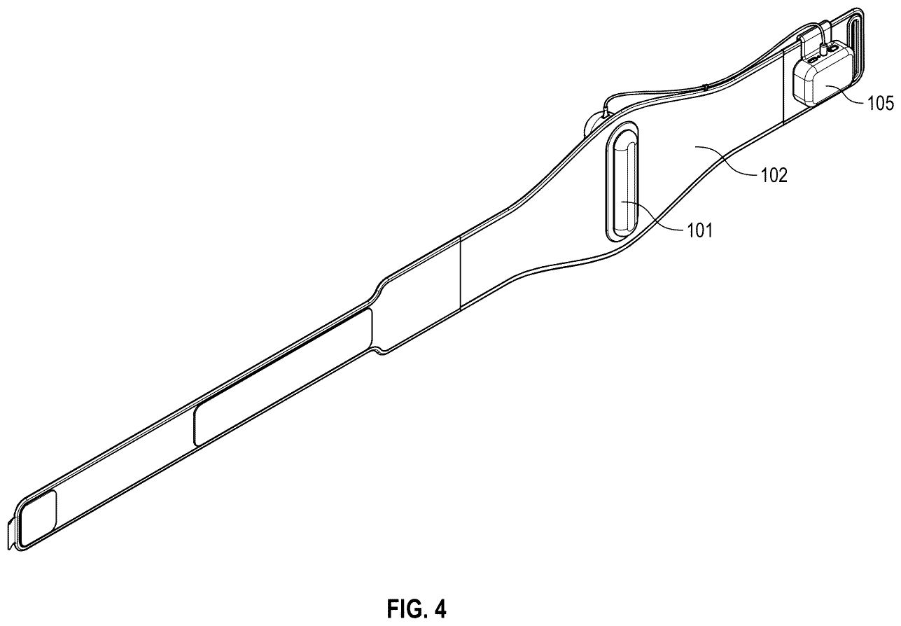

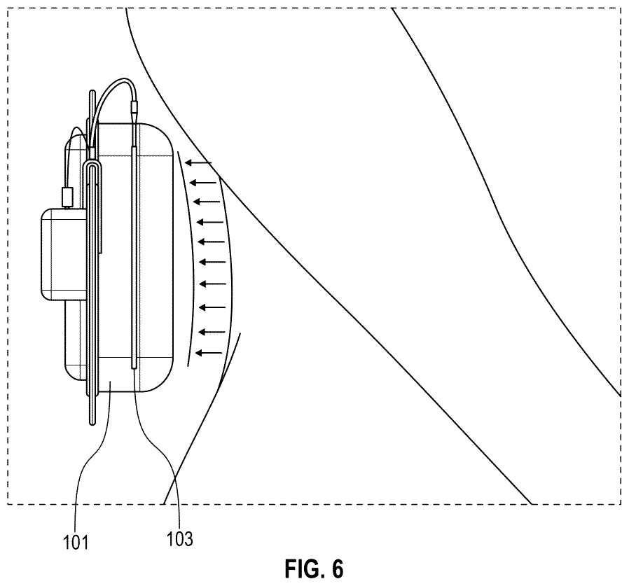

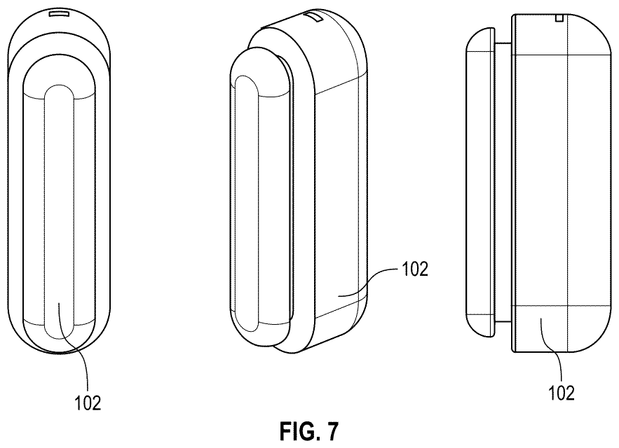

According to an embodiment of the present invention, an elastomer support assembly is disclosed. Referring FIG. 1, the assembly is intended for use in connection with a wearable device 102. The assembly includes an elastomer support 101 and a sensor 103 that is detachably integrated within the elastomer support 101. FIG. 7 shows different views of the elastomer support 101. In one aspect, the sensor 103 includes a position detection sensor 103. In another aspect, the sensor 103 includes a flex sensor, a strain sensor, and any other position and displacement detection sensor. FIG. 2 shows the wearable device 102 integrated with elastomer support assembly to be worn by a user. FIGS. 3 and 4 various view of the wearable device 102 integrated with elastomer support assembly. In this embodiment, the wearable device 102 is implemented as a belt. FIG. 6 shows the exact location of the placement of elastomer support 101.

In some aspects, the elastomer support 101 is made of flexible materials including foam, rubber and silicone. The foam includes open-cell foam and closed-cell foam.

The elastomer support 101 is attached to the wearable device 102 and adhered to a lumbar spine of a user when the wearable device 102 is worn by the user for allowing sensor 103 to detect lower back movement of the user. In some aspects, the lower back movement includes spine movement, low back movement, lumbar spine movement or various lordotic positions.

The elastomer support 101 is configured to replicate the position of the lower back of the user based on the change in user's position. This implies that the elastomer support 101 is flexible to conform to different body shapes without deformation such that the sensor 103 accurately detects and responds to movements of the user. In one embodiment, the elastomer support 101 is illustrated as having a specific size and shape; however, this is merely illustrative and should not be construed as limiting. The elastomer support 101 may be manufactured in various sizes to accommodate different user preferences, physical requirements, or other parameters. The elastomer support 101 is designed with versatility in both form and function, with the size and shape adjustable based on user needs or the intended application.

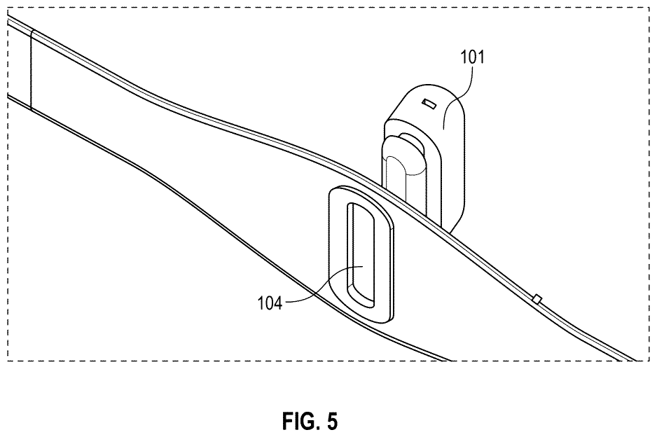

In one aspect, the wearable device 102 includes an opening 104, as shown in FIG. 5, into which the support is tightly attached. In a specific aspect, an electronic control unit (ECU) 105 is connected to the sensor 103 via a wire and is hooked on to the wearable device 102. The ECU 105 is configured to continuously transmit sensor data related to back posture of the user in real-time. The sensor data includes spine position data of the user. In various embodiments, the wearable device 102 includes a belt, a garment or any other body-wearable apparatus.

In one embodiment, the ECU 105 includes a data processing module (not shown), a vibration actuator (not shown) and a wireless communication module (not shown). The data processing module is configured to detect if the spine position of the user exceeds predefined thresholds. The predefined threshold includes initially calibrated spine position of the user. The initial calibration of spine position is performed using a software application in a mobile device 106. The vibration actuator is configured to receive control signal from the data processing module upon the data processing module detecting bad or poor position of user's spine and eventually alerts the user.

The wireless communication module is configured to communicate sensor data to the mobile application for real time visualization of user's back posture using a graphical user interface (GUI) of the mobile application.

In one embodiment, the elastomer support 101 may have the ECU 105 fully integrated within it. The ECU 105, along with its components, is designed to be securely housed within the elastomer support 101. The components of the ECU 105—including the data processing module, the vibration actuator, and the wireless communication module—are positioned within the elastomer support 101 to ensure both protection and optimal performance. The elastomer support 101 may take various shapes, such as cylindrical, cubical, circular, or square, and is adapted to allow the ECU 105 and its components to be built directly into its structure. The flexibility in design enhances the adaptability of the elastomer support 101.

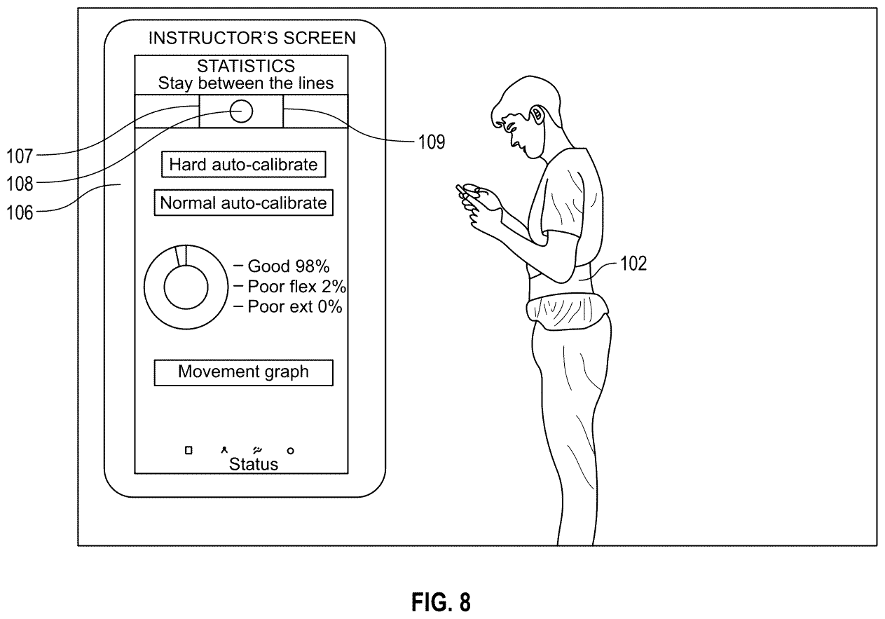

Referring FIG. 8, the user is allowed to visualize the back posture via the GUI which displays back posture through a digital sensor ball 107 and digital flexion 108 and extension barriers 109. The flexion 108 and extension barriers 109 are upper and lower limits respectively alerting the user of excessive movement. The flexion 108 and extension barriers 109 return to predetermined distances from the desired position upon recalibration.

According to another exemplary embodiment, the present invention disclosed herein is a wearable posture monitoring device, for detecting lower back movement of a user. In various aspects, the lower back movement may also be referred to as spine movement, low back movement, lumbar spine movement or various lordotic positions. The device comprises a body-wearable means having a first opening. In an exemplary aspect, the edges of the first opening are configured with a first frame made of plastic or silicone. The body-wearable means includes a belt, a garment or any other body-wearable means.

In various embodiments, the body-wearable means includes a belt, garment, or any other body wearable apparatus. Belt or garment type wearables provide flexibility in how the device can be worn, enhance user comfort and ensure consistent usage over extended periods. Other possible body-wearable means includes a vest, which for example houses sensing medium across the back and shoulders for comprehensive posture monitoring; a brace, providing for example additional support and ensuring sensor placement; adjustable straps worn for example over clothing; a harness for example to secure sensor placement during high-intensity activities; specially designed underwear for example for discreet monitoring; adhesive patches applied directly to the skin; a lightweight armature or exoskeleton; a specially designed backpack with integrated sensors; and arm or leg sleeves that incorporate sensors to monitor posture and related movements. Aforementioned body-wearable means enhance the flexibility and usability of the posture monitoring device, while catering to different preferences and activity levels.

The device further comprises a detachable support member accommodated in the first opening of the body-wearable means. The detachable support member includes first part, second part and third part. The third part of detachable support member is the central portion which contacts the first opening, i.e. the first frame surrounding the edges of the first opening as described earlier, when in use. When the support member is inserted into the first opening, the support member is held tightly within the first frame of the first opening. Due to high elasticity and appropriate moldability, the support member is easy to attach to the body-wearable means.

In an exemplary aspect, the detachable support member is made of fine-tuned foam, rubber, silicone or any other foam or material that works in a similar manner. In one aspect, the foam is open-cell foam while in another aspect, the foam is closed-cell foam. In some aspects, the sensor includes a posture detection sensor while also further includes at least one of a flex sensor, a strain sensor, and any other position and displacement detection sensor.

In one aspect, the detachable support member is formed with a tight hole in the center portion along the longitudinal axis of the support member. The device also comprises a detachable sensor which is designed to be accommodated in the hole of the detachable support member to detect the back posture, movement or position of a user. The sensor is configured to be inserted into the detachable support member such that the support member is adaptable to user's position, i.e. the support member is allowed to replicate or mimic the actions or movements of the low back of the user. In other words, the material used for detachable support member allows the support member to be adaptable more closely to the lumbar spine positioning for accurate detection of the user's posture by the sensor. Further comprised in the device is an electronic control unit (ECU) that is hooked or hanged on to the surface of the body-wearable means. In an exemplary aspect, the sensor is detachably connected with the ECU via a wire to alert the user upon detecting a change in the back posture of the user.

Upon integrating the sensor into the support member, inserting the support member into the first opening, and hooking or hanging the ECU on the body-wearable means, the body-wearable means is ready to be worn by the user. When the user wears the body-wearable means, the detachable support member contacts the lumbar spine region while allowing the sensor to indirectly contact the body of the user. The support member is flexible to conform to different body shapes without deformation such that the sensor accurately detects and responds to movements of the user.

In an exemplary embodiment, the flexibility of the support member allows the sensor to detect even the most subtle movements of the spine or lower back, while allowing for precise and accurate monitoring of the movements. In an aspect, the sensor detects the back position of the user accurately while indirect contacting the body of the user. In another aspect, the sensor is initially calibrated using a mobile application such that the sensor detects the back position of the user accurately while indirect contacting the body of the user.

In one embodiment, the device is implemented as a belt. The belt includes an elongated strap on one side. The strap is configured with Velcro modules through which the wearer is able to adjust the size and position of the belt, thereby ensuring a precise fit for every body type. Additionally, different lengths of this part of the belt are implemented to accommodate various percentiles. The Velcro modules include hook and loop modules along the elongated strap while the other side of the belt includes a small-sized strap having a second opening formed with a second frame similar to the first opening with the first frame. The second opening is configured to receive the strap which is adjustable using the Velcro modules. The second frame is made of silicone that prevents the fabric from tearing while also ensuring that the strap slides properly when the belt is adjusted. The straps are made of medium-elasticity fiber while the center part of the belt is formed of thick stretch spandex. This fabric allows increased adjustability to the body while helps adjusting the support member holding the sensor.

In one embodiment, the first and second frames are formed of co-injection molding, which method involves co-injecting both fabric and silicone to create a seamless integration. The fabric provides strength and flexibility, while the silicone ensures a secure and comfortable fit. This technique offers durability and a clean, finished appearance. In another embodiment, the first and second frames are formed of plastic. The plastic frames are sewn, snapped, or heat-stamped into place. These plastic frames provide a rigid structure that supports the silicone, while ensuring it maintains its shape and function over time. The choice of plastic material and method of attachment are adjustable to meet durability and cost requirements.

Once the device is worn by the user, the sensor automatically identifies the correct posture of the user, or by means of the initial calibration, which is discussed in the later part of the description. The sensor transmits posture data to the ECU, in real-time, upon detecting the change in the back posture of the user which is caused due to the physical of movement of the user. The ECU includes a data processing module which further comprises a processor and a memory. The data processing module communicates with the posture sensor to receive sensor data in real time. In one aspect, the data processing module employs algorithms to determine the poor position of the user's spine by receiving the posture sensor data. For example, data processing module may dimensionality reduction techniques (e.g., Principal Component Analysis (PCA), t-SNE, or autoencoders) to transform the high-dimensional sensor data into a lower-dimensional space. This transformation helps capture the essential features of the spinal position while reducing noise and computational complexity. The reduced-dimensional data can then be combined to create a simplified yet comprehensive representation of the current spinal position. The data processing module may evaluate whether the current spinal position exceeds predefined thresholds. If the deviation exceeds these thresholds, a control signal may be generated and sent to activate an vibration actuator for providing immediate haptic feedback to the user.

The ECU includes the vibration actuator to alert the user upon detecting the change in the back posture of the user. In one aspect, the data processing module transmits the sensor data received from the posture sensor to the vibration actuator, for example as a control signal, when the data processing module detects a bad or poor position of the user's spine. Upon receiving the control signal, the vibration actuator vibrates to alert the user for correcting the posture. The vibration actuator is preferably a component or a device that generates vibrations to provide haptic feedback to the user. Examples of vibration actuators include Eccentric Rotating Mass (ERM) motors, which use an off-center mass to create vibrations; Linear Resonant Actuators (LRA), which utilize a moving mass and electromagnetic coil for precise feedback; Piezoelectric actuators, which generate vibrations through the deformation of piezoelectric materials when an electric field is applied; and Voice Coil Actuators, which operate through the interaction of a magnetic field and electric current. These actuators can be seamlessly integrated into ECU to provide haptic feedback to the user.

A poor position of the spine, as disclosed herein, refers to any posture, movement or position, that deviates from the natural, initially calibrated position the spine. The poor position of the spine might include excessive slouching, overextension, flexion, or any misalignment that puts undue stress on the spinal column. These poor positions vary depending on the activity or situation. For example, a poor position while standing can differ from that of while sitting, performing squats, lifting weights, doing sit-ups, or practicing yoga.

The ECU also includes an optional wireless communication module to communicate sensor data to a mobile device having a mobile application for real time visualization of user's back posture using a graphical user interface (GUI) of the mobile application.

In some aspects, the mobile device is referred to as a smart device. The smart device includes an electronic device that is typically connected to other devices or networks via various wireless protocols such as Bluetooth, Wi-Fi, or cellular networks. The smart device can operate interactively and autonomously. In the context of the posture monitoring device, the smart device includes a smartphone, laptop or tablet that may run a mobile application which is designed to interface with the monitoring device.

Using the GUI of the mobile application, the user is able to visualize the back posture. The GUI displays the back posture through a digital sensor ball and digital flexion and extension barriers. The flexion and extension barriers are upper and lower thresholds respectively alerting the user of excessive movement. The graphical representation of the thresholds and the current posture of the spinal position allows the user to intuitively understand their posture and maintain the correct spinal position with ease.

The mobile application includes a calibration module for calibrating or recalibrating the sensor based on desired position of the user. The flexion and extension barriers return to predetermined distances from the desired position upon calibration or recalibration.

The dynamic nature of the device ensures that the device can be worn during various exercises or daily activities without requiring the user to adjust or alter the device's arrangement. This adaptability improves the device's usability and effectiveness, as it seamlessly integrates into the user's routine while consistently providing accurate posture data.

It will finally be understood that the disclosed embodiments are presently preferred examples of how to make and use the claimed invention, and are intended to be explanatory rather than limiting the scope of the invention as defined by the claims below. Reasonable variations and modifications of the illustrated examples in the foregoing written specification and drawings are possible without departing from the scope of the invention as defined in the claim below. It should further be understood that to the extent the term “invention” is used in the written specification, it is not to be construed as a limited term as to number of claimed or disclosed inventions or the scope of any such invention, but as a term which has long been conveniently and widely used to describe new and useful improvements in technology. The scope of the invention supported by the above disclosure should accordingly be construed within the scope of what it teaches and suggests to those skilled in the art, and within the scope of any claims that the above disclosure supports. The scope of the invention is accordingly defined by the following claims.

This application is intended to cover any adaptations or variations of the present invention. Therefore, it is manifestly intended that this invention be limited only by the claims and the equivalents thereof.

1. An elastomer support assembly for use in connection with a wearable device, comprising:
an elastomer support;
a sensor detachably integrated within the elastomer support;
wherein the support is attached to the wearable device and adhered to a lumbar spine of a user when the wearable device is worn by the user for allowing sensors to detect lower back movement of the user.

2. The elastomer support assembly of claim 1, wherein the support is tightly attached to the wearable device in an opening.

3. The elastomer support assembly of claim 1 further comprises an electronic control unit (ECU) which is connected the sensor via a wire to continuously transmit sensor data related to back posture of the user in real-time.

4. The elastomer support assembly of claim 3, wherein the ECU is hooked on to the wearable device.

5. The elastomer support assembly of claim 1, wherein the lower back movement includes spine movement, low back movement, lumbar spine movement or various lordotic positions.

6. The elastomer support assembly of claim 1, wherein the elastomer support replicates the position of the lower back of the user based on the change in user's position.

7. The elastomer support assembly of claim 1, wherein the elastomer support is made of flexible materials including foam, rubber and silicone.

8. The elastomer support assembly of claim 7, wherein the foam is open-cell foam and closed-cell foam.

9. The elastomer support assembly of claim 1, wherein the sensor includes a position detection sensor.

10. The elastomer support assembly of claim 1, wherein the sensor includes a flex sensor, a strain sensor, and any other position and displacement detection sensor.

11. The elastomer support assembly of claim 1, wherein the elastomer support is flexible to conform to different body shapes without deformation such that the sensor accurately detects and responds to movements of the user.

12. The elastomer support assembly of claim 1, wherein the wearable device includes a belt, a garment or any other body-wearable apparatus.

13. The elastomer support assembly of claim 3, wherein the sensor data includes spine position data of the user.

14. The elastomer support assembly of claim 3, wherein the ECU includes a data processing module to detect if the spine position of the user exceeds predefined thresholds.

15. The elastomer support assembly of claim 14, wherein the predefined threshold includes initially calibrated spine position of the user.

16. The elastomer support assembly of claim 15, wherein the initial calibration of spine position is performed using a software application in a mobile device.

17. The elastomer support assembly of claim 14, wherein the ECU includes a vibration actuator that receives control signal from the data processing module upon the data processing module detecting bad or poor position of user's spine and eventually alerts the user.

18. The elastomer support assembly of claim 3, wherein the ECU includes a wireless communication module to communicate sensor data to the mobile application for real time visualization of user's back posture using a graphical user interface (GUI) of the mobile application.

19. The elastomer support assembly of claim 18, wherein the GUI visualizes the user's back posture by displaying back posture through a digital sensor ball and digital flexion and extension barriers.

20. The elastomer support assembly of claim 19, wherein the flexion and extension barriers are upper and lower limits respectively alerting the user of excessive movement.

21. The elastomer support assembly of claim 20, wherein the flexion and extension barriers return to predetermined distances from the desired position upon recalibration.

22. The elastomer support assembly of claim 3, wherein the ECU is housed within the elastomer support.

23. The elastomer support assembly of claim 3, wherein the components of ECU including the data processing module, the vibration actuator and the wireless communication module are integrated within the elastomer support.
